(19) United States
(12) Patent Application Publication
(10) Pub. No.: US 2010/0210922 A1
(43) Pub. Date: Aug. 19, 2010

(54) IMPLANTABLE SOUND GENERATOR AND SYSTEM AND METHOD FOR THE DETECTION AND ANALYSIS OF PROCESSES AND CONDITIONS

(76) Inventor: Bernhard Clasbrummel, Bochum (DE)

Correspondence Address: Kubotera & Associates, LLC, 200 Daingerfield Rd, Suite 202, Alexandria, VA, 22314, US

(21) Appl. No.: 12/666,336

(22) PCT Filed: Jun. 26, 2008
(86) PCT No.: PCT/EP08/05194

§ 371 Date: Apr. 1, 2010

(30) Foreign Application Priority Data
Jun. 28, 2007 (DE) 10 2007 030 270.5

(51) Int. Cl. A61B 5/00

(52) U.S. Cl. 600/301

(57) ABSTRACT

Implantable sound generator for generating sound from movements and/or forces and/or pressure in the human or animal body, for the detection of physiologic and/or non-physiologic and/or pathologic processes in interaction with an acoustic receiver unit. The sound generator consists of a bio-degradable material and has a miniaturised configuration. The sound generator may be configured in the form of a whistle or a pulse sensor or a chim or ratchet or as sonic thermometer. The sound generator may be used to detect and analyse the pulse and/or the blood flow and/or movements of the cardiac wall and to detect and analyse loaded bones and/or capsular tissue and/or impolants and/or prostheses and/or materials for the osteosynthesis of bones and/or the temperature. The invention moreover relates to a system for detecting and analysing physiologic and/or non-physiologic and/or pathologic processes by means of at least one sound generator and at least one acoustic receiver unit, as well as to a method of detecting and analysing non-physiologic and/or pathologic processes by means of at least one sound generator and at least one acoustic receiver unit.

The present invention relates to a sound generator for implantation as well as to a system and a method for the detection and analysis of processes and conditions, in particular for and in the human and animal body.

Numerous implantable sensors for the detection of conditions and processes in the human body have become known in prior art, which emit expediently, also in wireless technology, signals to receiving and analyser units. An active implantable sound generator is known from the U.S. Pat. No. 3,672,352A, which cooperates with a source of energy which is equally envisaged for implantation together with the sound generator, in a disadvantageous manner.

From the document WO 2005/102151, for instance, a passive sensor with wireless transmission is known which is detected by ultrasound by means of an active analyser unit. As a matter of fact, however, passive sensors can only be interrogated and hence the analyser unit must interrogate the sensor continuously, with a substantial consumption of energy, in order to ensure continuous monitoring of the sensor. This is laborious and complex as well as expensive and particularly in the field of medicine, this may result in problems, inconvenience and side effects in treatment and diagnosis.

The present invention is therefore based on the object of providing an improved implantable sensor and an improved method for the detection and analysis of conditions and processes in the human and animal body, with active transmission of signals from the sensor unit to a receiver and/or analyzer unit.

The object is solved with the coordinated claims. Preferred embodiments of the present invention are implemented with the features defined in the dependent claims and/or by features mentioned in the description.

In a particularly expedient form using an implantable sound generator for the generation of sound from movements and/or forces and/or pressures in the human or animal body, an inventive implantable sensor is provided for the detection of physiologic and/or non-physiologic and/or pathologic processes, which interacts with an acoustic receiver unit disposed outside the human or animal body. According to this concept, an inventive implantable sound generator presents an expediently miniaturized configuration and is made of a bio-degradable material.

An inventive sound generator may be expediently designed as miniaturized whistle, in particular, which produces whistling tones, noise and clicks in the manner of communicating dolphins and whales whose sound waves emitted are particularly well propagated in a fluid medium. In a particularly expedient form, the inventive whistle may comprise an air stream generator, a capillary system, a whistling-tone unit and a resonant cavity which are each so configured and disposed that the bending of the whistle generates a strong air flow that creates a defined whistling or clicking sound. In particular, in accordance with the invention, the air stream generator may expediently include a cavity with a volume-varying mechanism, which operates in response to movements and consists of a plurality of lever arms which are so disposed that a diaphragm results in a volume compression of the cavity, with the cavity of the air stream generator being filled with a non-compressible liquid. A discharging tube is appropriately joined to the cavity of the air stream generator on the downstream side, which opens into a capillary system that is follows by a whistling-tone unit, with the capillary system being expediently configured such that it becomes progressively more and more hydrophobic in a direction towards the whistling-tone unit whilst its zone in the vicinity of the whistling-tone unit is designed to be hydrophobic in such a way that it produces the effect of a liquid barrier. For the transmission of the sound waves generated by the whistling-tone unit, the whistling-tone unit suitably opens into a resonant cavity that presents an appropriate configuration in order to transmit the sound waves generated by the whistling-tone unit efficiently into the human body.

In accordance with another expedient embodiment of the present invention, the inventive sound generator may be configured as pulse sensor, with a tensioned diaphragm being mechanically caused to vibrate by means of an appropriate means. In that configuration, the pulse sensor suitably comprises an exterior shell and a first tensioned diaphragm and a second diaphragm on the pulse side, which is stretched over a flexible wall reinforcement, with a lever being disposed on the diaphragm on the pulse side and interacting with a clapper disposed inside the exterior shell in such a way that in case of pulsation, the lever operates the clapper in such a way that the clapper hits against the first tensioned diaphragm, generating a sound. Such a sound generator configured as pulse sensor in accordance with the present invention is particularly well suitable for measuring the blood flow on blood vessels, vessels or on the heart. In correspondence with a modification of the aforedescribed second embodiment of the present invention, the pulse sensor may comprise a tensioned string disposed inside the exterior shell, which interacts with the lever and which the lever causes to vibrate. The pulse sensor with the second diaphragm on the pulse side is particularly expediently disposed on a blood vessel or on a tissue wall inside the human body.

In accordance with another expedient embodiment of the present invention, the inventive sound generator is appropriately configured as chime or ratchet, with the chime or ratchet expediently generating a sound in a semi-hydraulic manner by hydraulically driving a mechanical element and by the provision of friction elements and/or a biased diaphragm for mechanical generation of a sound. In this configuration, an inventive sound generator configured as chime or ratchet expediently comprises a biased elongate tube that is filled with a non-compressible liquid, with a semi-hydraulic element being so disposed and configured on the tube that a ratchet, which is connected to the joint, creates a sound when the volume of the tube is reduced in a resonant cavity in which the ratchet is disposed; here, the ratchet comprises appropriately a lever with a lever end that rattles over projections formed inside the resonant cavity when the joint is activated. In this configuration it is expedient to dispose a restoring sprig in the resonant cavity and connect it to the joint in such a way that when the pressure in the tube is reduced the position of the ratchet inside the resonant cavity is restored. Alternatively or in addition to the rattling movement of the lever end over projections, the lever end may also expediently hit against a tensioned diaphragm provided inside the resonant cavity. An inventive sound generator configured as chime or ratchet may be integrated into prosthesis in a particularly advantageous manner, in which case tensions occurring upon a load on the prosthesis may be detected and a digital quality assurance after the prosthesis implantation analysis will become possible to detect loosening of prostheses. Such an inventive sound generator configured as chime or ratchet may furthermore be integrated, in a particularly expedient form, into prostheses components such as femoral heads, acetabula or components of knee or shoulder prostheses or it may be mounted on implants for the osteosynthesis of bones such as plates, nails and screws; in these cases the healing of fractures becomes detectable and can be monitored in an expedient manner so that the loading, e.g. of legs after fractures, in correspondence with the stage of healing will be possible, which results expediently an a shorter healing period or in the early detection of delayed healing processes. In this manner, it becomes moreover expediently possible to monitor the behaviour of implants and hence progress in the healing process in emergency surgery or orthopaedics.

In correspondence with a further advantageous embodiment of the present invention, an inventive sound generator may be expediently configured as sonic thermometer which includes suitably a memory material that interacts with a diaphragm, with the creation of a sound as a function of temperature. With such an inventive sound generator, which is expediently configured as sonic thermometer, it is possible to provide, in an expedient manner, a temperature-controlled tumour therapy (control of tumour heating in a magnetic field), control of the body temperature (contraception), measurement of the heating of regenerating tissue (bones, tendons) or investigation into the behaviour of animals with relation to the body temperature.

The present invention will be described in details in the following with reference to accompanying schematic drawings in which:

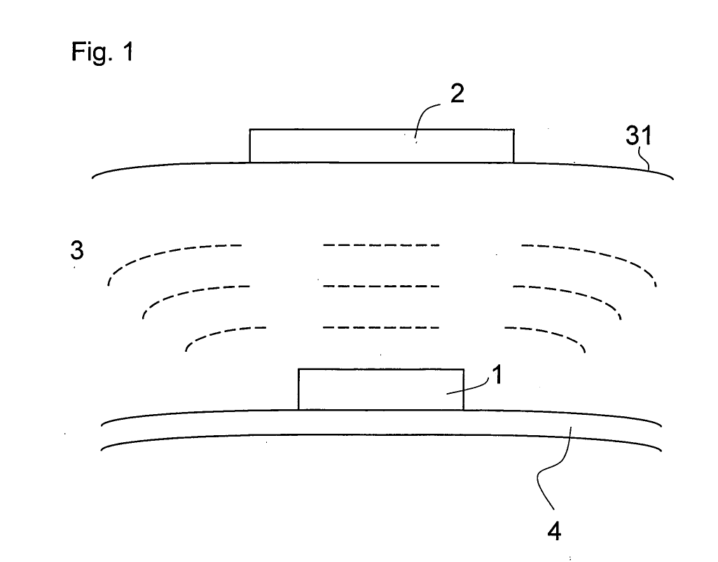

FIG. 1 is a schematic view of an inventive sound generator including an acoustic receiver unit, a schematic representation of the inventive system for the detection and analysis of physiologic and/or non-physiologic and/or pathologic processes, as well as a schematic illustration of the corresponding inventive method;

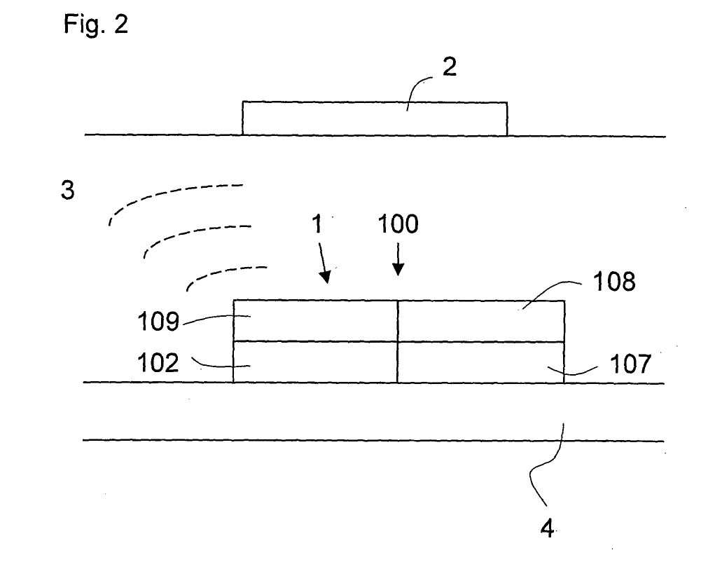

FIG. 2 is a schematic representation of an inventive sound generator in accordance with a first embodiment of the present invention;

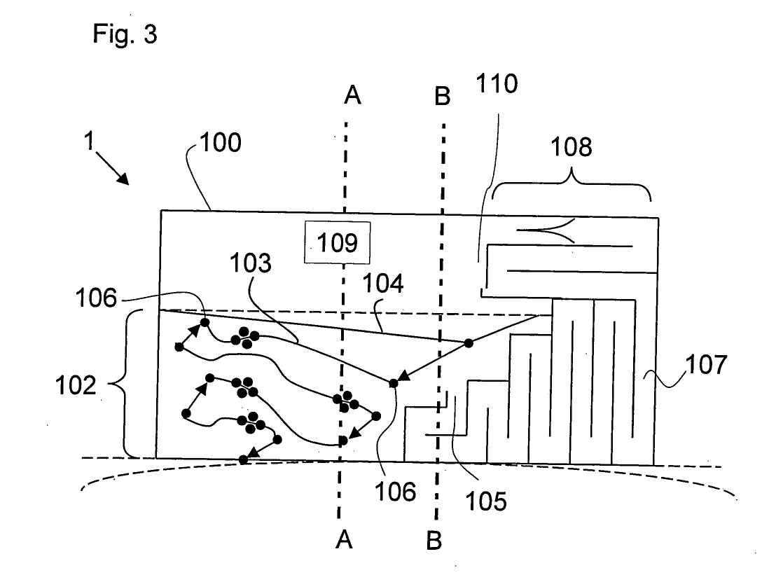

FIG. 3 shows an enlarged cross-sectional view of the sound generator represented in FIG. 2;

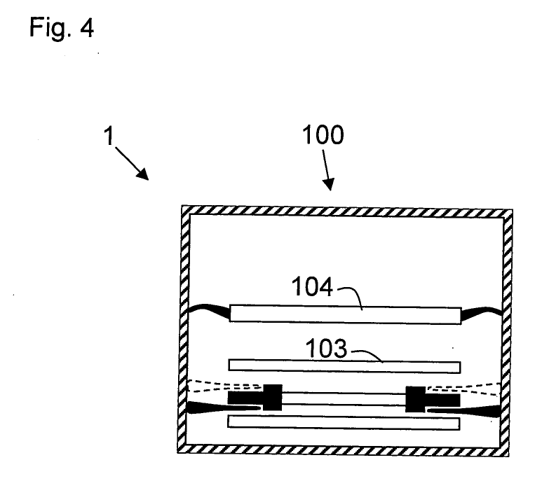

FIG. 4 is a section taken through the sound generator according to FIG. 3 along the line A-A in FIG. 3;

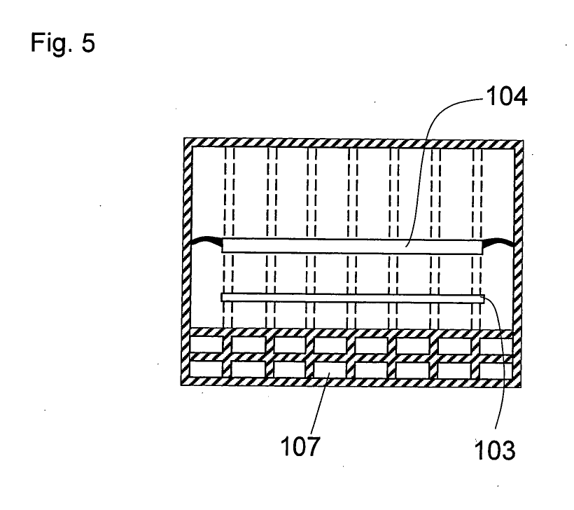

FIG. 5 shows a section through the sound generator according to FIG. 3 along the line B-B in FIG. 3;

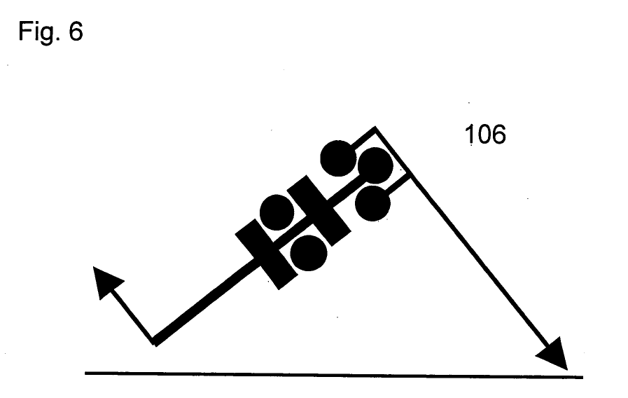

FIG. 6 is an enlarged detail view of the sound generator according to FIG. 3;

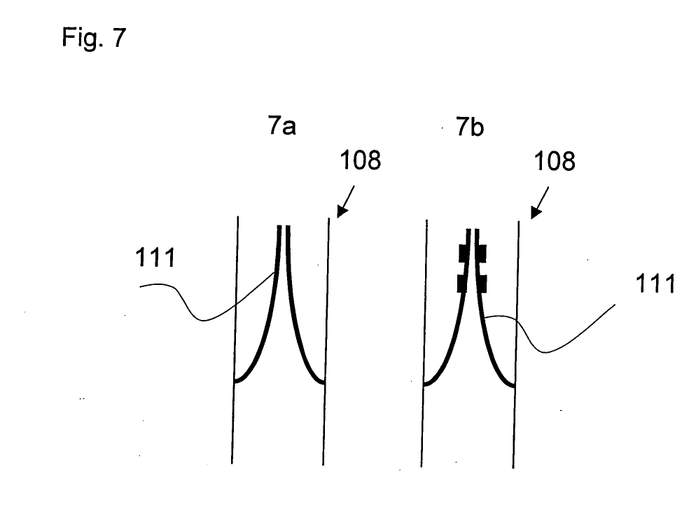

FIGS. 7A and 7B are each other enlarged partial views of the sound generator according to FIG. 2;

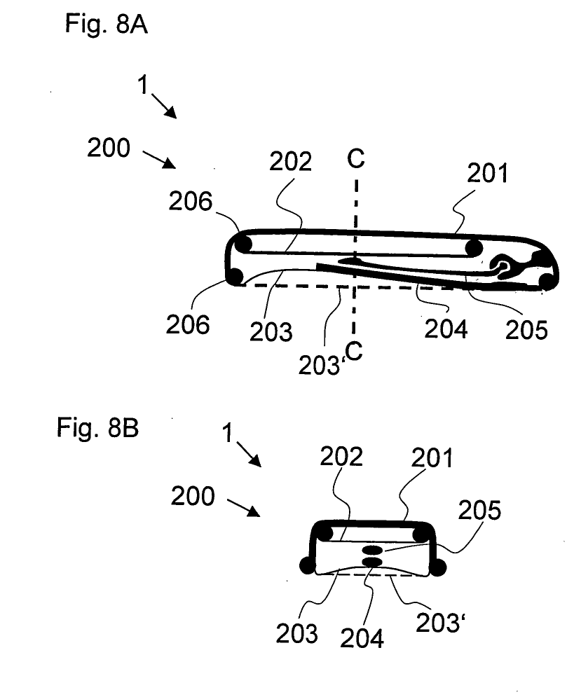

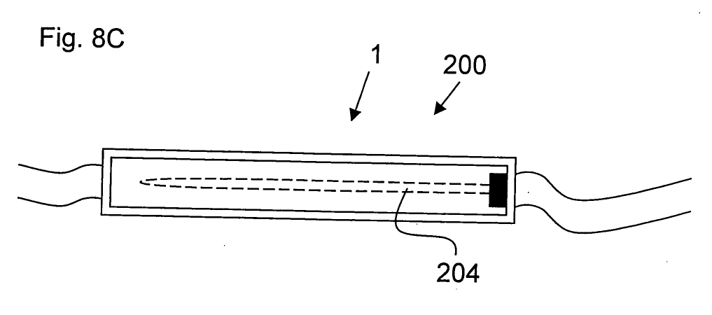

FIG. 8A shows a schematic section through a sound generator in correspondence with another embodiment of the present invention;

FIG. 8B is a section taking through the line C-C in FIG. 8A;

FIG. 8C is a schematic plan view of the sound generator according to FIG. 8A;

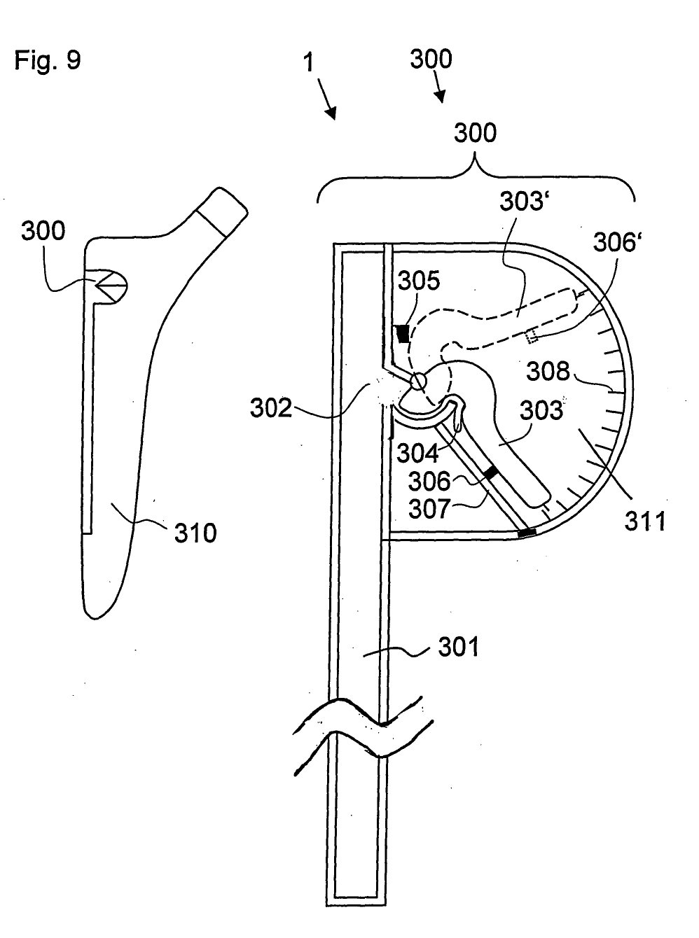

FIG. 9 shows a section through a sound generator in correspondence with a further embodiment of the present invention and a schematic illustration of a sound generator integrated into a prosthesis;

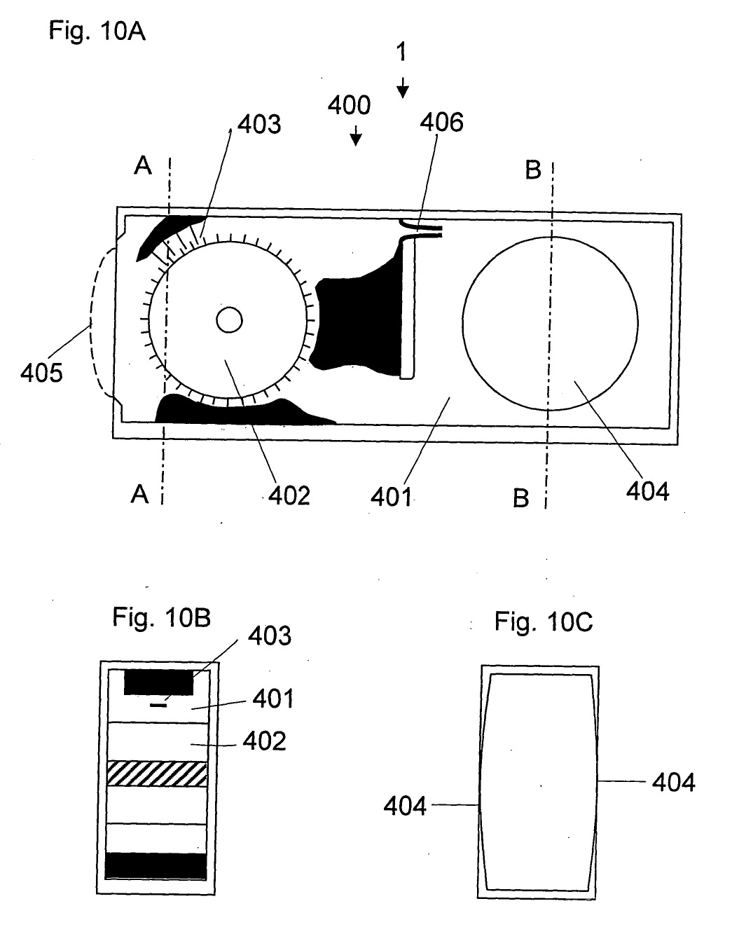

FIG. 10A is a section through a sound generator according to another embodiment of the present invention;

FIG. 10B is a section through the sound generator according to FIG. 10A along the line A-A in FIG. 10A;

FIG. 10C shows a section through the sound generator according to FIG. 10A along the line B-B in FIG. 10A;

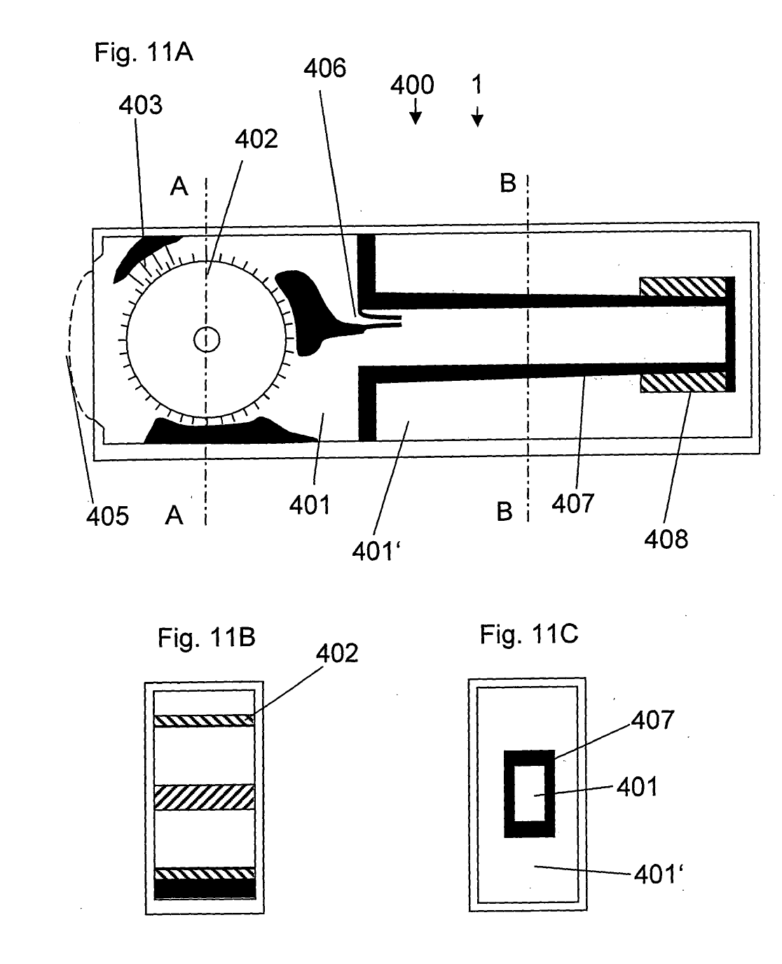

FIG. 11A is a section through a sound generator in correspondence with a further embodiment of the present invention;

FIG. 11B is a section through the sound generator according to FIG. 11A along the line A-A in FIG. 11A;

FIG. 11C is a section through the sound generator according to FIG. 11A along the line B-B in FIG. 11A;

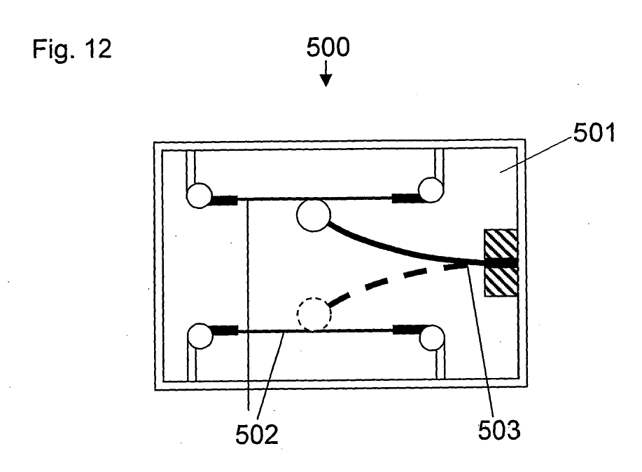

FIG. 12 shows a schematic view of a sound generator in accordance with another embodiment of the present invention, and

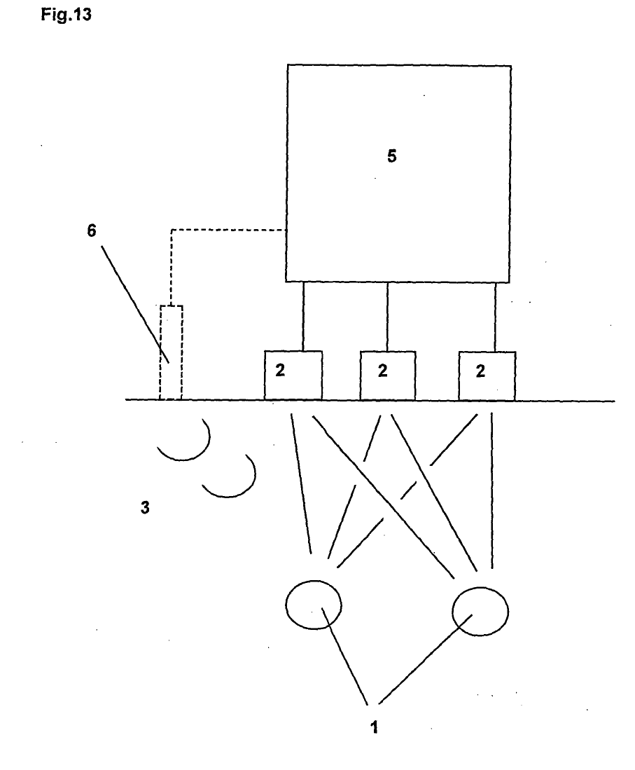

FIG. 13 is a schematic illustration of one embodiment of the inventive method and of the inventive system for the detection and analysis of conditions and processes in the human or animal body.

FIG. 1 shows a schematic view of an inventive sound generator 1 comprising an acoustic receiver unit 2 that is suitably disposed inside the human or animal body, wherein the acoustic receiver unit 2 is appropriately arranged outside the human or animal body such that it receives signals emitted from the sound generator 1 and transmits them on to an analyser unit which analyses and stores and documents the signals in an appropriate manner. In accordance with the invention, the sound generator unit 1, the acoustic receiver unit 2 and the analyser unit, which is not illustrated in FIG. 1, interact with each other in such a way that signals are detected and received in a digital form and that the received frequency spectrum is analysed expediently via Fourier transforms, for instance. In a suitable and expedient manner, it is also possible that at least two sound generators 1 are implanted in an appropriate geometric arrangement and/or that at least two acoustic receiver units are disposed on the surface of the body to be examined, equally in a suitable geometric arrangement, so that an advantageous analysis will be possible also via model computations and/or with exploitation of Doppler effects, for example. When in such a system one sensor is arranged, for example, in front of a vessel and a second sensor behind the vessel it is possible and expedient, for instance, to perform a quantitative measurement of the flow by means of a pressure or acceleration sensor, with the signal in front of the vessel remaining invariable whereas it is modified behind the vessel on account of Doppler effects occurring because the acoustic signal must pass through the blood vessel with its corpuscular constituents. In such an array, for instance, at least two—and expediently six—acceleration or pressure sensors permit the measurement of the flow around a vessel. With two or more acoustic receivers spaced from each other by a defined predetermined distance, it is possible to locate a vessel and to perform localisation by triangulation, for example. It is self-evident that in such a case a receiver unit is so configured that it is capable of reading out several inventive sound generators at the same time. An exemplary advantageous embodiment of the arrangement of the sound generator 1 and the acoustic receiver unit 2 will be described in the following with reference to the drawing No. 13.

FIG. 2 illustrates an enlarged schematic view of an inventive sound generator 1 disposed inside the human body and an acoustic receiver unit 2 arranged on the body surface, it being clear that a microphone or hydrophone may be used in particular as suitable acoustic receiver unit 2. The inventive sound generator 1 may be configured as whistle 100—which is particularly advantageous—which whistle comprises an air stream generator 102, a capillary system 107, a whistling-tone unit 108 and a resonant cavity 109.

FIG. 3 illustrates another enlarged schematic view of an inventive sound generator 1 configured in the form of a whistle 100 whilst FIGS. 4 and 5 show each sectional views of the sound generator 1 according to FIG. 3 along the line A-A or the line B-B of FIG. 3, respectively. The air stream generator 2 comprises a volume-varying mechanism responsive to bending, which consists of lever arms 103 that are disposed inside a volume enclosed by a partially resilient diaphragm 104 and filled with a liquid and that interact with the partially resilient diaphragm 104. As a result of an expedient connection of successive lever arms 103 it is possible to adapt a predetermined reduction of the volume in correspondence with bending of the inventive sound generator 1 within defined limits. One example of an articulated connection 106 of the lever arms 103 is schematically illustrated in FIG. 6.

At least one discharging tube 105 is joined to the space with the lever arms 103, which is filled with a liquid, on the downstream side, which tube opens into a capillary system 107 that is equally joined by a downstream whistling-tone unit 108. When the sound generator 100 is bent the diaphragm 104 activates the lever arms 103 in the volume filled with a liquid, so that the liquid acts upon the capillary system 107 through the discharging tube 105; the capillary system presents a progressively more and more hydrophobic configuration and has a strongly hydrophobic design in the vicinity of the whistling-tone unit 108 so that liquid cannot arrive there and a strong air stream is generated to act upon the liquid when pressure is exerted, which air stream activates the whistling-tone unit that is configured as whistle 108 or rattle 108, which is schematically illustrated in an enlarged view in FIGS. 7A and 7B. It is self-evident that the hydrophobic sound-generating zone may also consist of more than a single whistle, which whistles are expediently so configured that they produce tones of different frequencies. The whistling-tone unit 108 may be composed of lips 111 of different densities, for instance and in an advantageous form, which are provided with different micro weights, if necessary. The air stream downstream of the whistling-tone unit 108 opens into the resonant cavity 109, with a valve 110 being provided in the vicinity of the whistling-tone unit on the capillary system 107 so that when the sound generator 1 is bent in the reverse direction air flows back into the capillary system 107. It is self-evident that in other expedient embodiments the air stream generator 102 may also be operated without liquid filled into the volume and that in these embodiments, on account of the missing liquid charge; there are no particular demands on the inner implant surfaces with respect to the wetting properties (hydrophilic characteristics).

For a better comprehension of the present invention, some model computations were made to this end, which are also illustrated in a tabular form.

By bending an air stream generator 102 (fastened to a bone under a natural load) having a length of roughly 12 mm, a height of 4 mm and a width of 5 mm, one can achieve a liquid stream of roughly 2 mm in a capillary tube 107 having a thickness of 100 μm. On account of the inventive lever arm system 103, it is possible then to achieve a reinforcing mechanism that results in a velocity of the displacing liquid/air front in a micro tube, which is 10 to 100 times the original speed.

In a micro tube having a thickness of 0.1 to 0.3 to 0.3 m, one should achieve, with the air stream generator 102, an air stream of a velocity between 300 and 3000 km/h on a micro whistle, so that one should obtain a whistling tone. Table 1 illustrates exemplary computations of air flows in capillary tubes having a thickness of 0.1 or 0.3 mm, with subsequent whistling being created.

TABLE 1
Tabular survey of the air velocities in the case of amplification of the
air stream in capillary tubes 107 by the air stream generator 102 (LG). Orienting
logarithmic amplifications induced by different generators LG were assumed. An air
stream of supersonic velocity only is capable of producing noise at a whistle (grey
boxes).

| | | Diameter | | | Air flow | | | Amplified | | Amplified | Air flow |
|---|---|---|---|---|---|---|---|---|---|---|---|
| | Amplification | of | Motion of | | in the | Amplified air | Air flow | air flow | Air flow | air flow | peak at |
| Air flow | by | capillary | the air | | capillary | flow at | peak at | at | peak at | at | whistle |
| generator | air flow | [symbol] | column | | [symbol] | [symbol] | whistle I | [symbol] | whistle II | [symbol] | III |
| (LG) | [symbol] | mm | mm | sec | Km/h | factor | Km/h | factor | Km/h | factor | Km/h |
| Without LG | 0 | 0.10 | 2.0 | 0.10 | 0.072 | 50 | 3.6 | 150 | 10.8 | 500 | 36.0 |
| With LG | 0 | 0.30 | 0.7 | 0.10 | 0.024 | 50 | 1.2 | 150 | 3.6 | 500 | 12.0 |
| LG A | 3 | 0.10 | 6.0 | 0.10 | 0.216 | 50 | 10.8 | 150 | 32.4 | 500 | 108.0 |
| LG A | 3 | 0.30 | 2.0 | 0.10 | 0.072 | 50 | 3.6 | 150 | 10.8 | 500 | 36.0 |
| LG B | 10 | 0.10 | 20.0 | 0.10 | 0.720 | 50 | 36.0 | 150 | 108.0 | 500 | [symbol] |
| LG B | 10 | 0.30 | 6.7 | 0.10 | 0.240 | 50 | 12.0 | 150 | 36.0 | 500 | 120.0 |
| LG C | 30 | 0.10 | 60.0 | 0.10 | 2.160 | 50 | 108.0 | 150 | [symbol] | 500 | [symbol] |
| LG C | 30 | 0.30 | 20.0 | 0.10 | 0.720 | 50 | 36.0 | 150 | 108.0 | 500 | [symbol] |
| LG D | 100 | 0.10 | 200.0 | 0.10 | 7.200 | 50 | [symbol] | 150 | [symbol] | 500 | [symbol] |
| LG D | 100 | 0.30 | 66.7 | 0.10 | 2.400 | 50 | 120.0 | 150 | 360.0 | 500 | 1200.0 |

[symbol]

indicates data missing or illegible when filed

Volumetric flow=volume/time=distance·cross-sectional area/time
=distance/time·cross-sectional area
=velocity·cross-sectional area
=v·A=constant

[symbol]

v₁·A₁=v₂·A₂=v₃·A₃ etc.

[symbol]

v₂·A₁/A₂·v₁

Conclusion: For an increase of the velocity v₁ by the factor of 100 the cross-sectional area A₂ must be reduced to 1/100 relative to A₁: with A₁=d₁²·π/4 and A₂=d₂²·π/4.

[symbol]

v₂=(d₁/d₂)²·v₁
[symbol] from a reduction of the diameter d₂ to 1/10 of d₁ derives that v₂ is 100 times greater than v₁
[symbol] with a reduction to 1/100=>v₂ is 10,000 times greater than v₁
[symbol] The factor of ten thousand is reached if the diameter is reduced, for instance, from 1 mm=1000 μm to 10 μm.

FIG. 8A illustrates a schematic view of a sound generator 1 configured as pulse sensor 200 in accordance with the invention, while FIG. 8B is a cross-sectional view taken through the sound generator 1 in FIG. 8A along the line C-C in FIG. 8A and FIG. 8C shows a schematic view of the pulse sensor 200 disposed on a blood vessel, which illustrated in FIGS. 8A and B. The inventive pulse sensor 200 comprises an exterior shell 201 inside which a first diaphragm 203, 203′ is disposed, wherein the exterior shell 201 is closed at the bottom by a second diaphragm 203, 203′. In this configuration, the second diaphragm 203, 203′ on the pulse side is suitably stretched over a flexible wall reinforcement, with a lever 204 being disposed on the diaphragm 203, 203′ and interacting with a clapper 205 arranged inside the exterior shell 201 in such a way that in the case of pulsation the lever 204 operates the clapper 205 in such a way that the clapper 205 hits against the first tensioned diaphragm 202, thus generating a sound.

FIG. 9 is a further schematic illustration of an inventive sound generator 1, which is expediently configured as chime and/or ratchet 300, together with another schematic view of prosthesis. In accordance with the invention, the inventive chime and/or the ratchet 300 generate a sound in a semi-hydraulic manner, by driving a mechanical element hydraulically, which produces mechanically a sound via the provision of friction elements 308 and/or a biased diaphragm 307. In this configuration, the chime and/or the ratchet comprise appropriately a lever 303, 303′ with a lever end which, when a joint 302 is activated, rattles over projections 308 disposed inside a resonant volume 311, and/or a stop 306, 306′ hitting against the diaphragm 307. Inside the resonant volume 311, a restoring spring 305 is appropriately disposed such that, when the pressure in the tube 301 filled with a liquid is reduced, the position of the ratchet 303, 303′ inside the resonant volume 311 is restored. The flexible tube 301 in this configuration is filled with a non-compressible liquid so that bending of the tube 301 results in activation of the joint 301 and in a movement of the lever 303, 303′.

FIG. 10 illustrates a schematic view of a modified embodiment of the chime/ratchet 400 illustrated in FIG. 9; FIGS. 10B and 10C show each schematic sectional views of the ratchet 400 shown in FIG. 10A, taken along the lines A-A or B-B in FIG. 10A, respectively. FIGS. 11A, B and C additionally show a modified embodiment of the ratchet 400 in FIG. 10. The ratchet 400 in the embodiment according to FIG. 10 comprises a cavity 401 filled with a liquid, in which a gear wheel 402 is disposed whose teeth rattle over projections 403 suitably arranged in the cavity 401 when, on account of bending of a diaphragm 404 equally disposed in the cavity 401, the liquid drives the gear wheel 402 that presents a configuration similar to the design of a blade wheel, with a resilient equalising diaphragm 405 being disposed in a suitable manner on the wall in the space behind the gear wheel 402, into which liquid flows, too. For the return flow of the liquid into the vicinity of the diaphragm 404, the inventive ratchet 400 moreover comprises an appropriately disposed valve 406. In the embodiment shown in FIG. 10, the resilient diaphragm 404 is responsive to pressure and exerts a pressure onto the liquid present in the cavity 401 when the sound generator 1 is bent; the resilient diaphragm 404 may be expediently configured as a cylinder whose axis extends in parallel with the axis of the blade wheel-like gear wheel 402. In this embodiment, the resilient diaphragm 404 in the embodiment according to FIG. 10 is bent when the pressure in the body is increased, therefore exerting pressure on the liquid present in the cavity 401, with the diaphragm 404 being possibly configured as square cylinder having two diaphragms 404 in an appropriate form, whose axis extends in parallel with the axis of the blade wheel-like gear wheel 402.

FIGS. 11A, B and C illustrate an expedient modification of the ratchet 400 according to FIG. 10, which comprises a first cavity 401 filled with a liquid and a second cavity 401′ which may equally be filled with a liquid and/or with a gas (air); in this embodiment, a gear wheel 402 similar to a blade wheel and projections 403 are disposed, too, in the first cavity 401, like in the embodiment according to FIG. 10, whilst the cavity 401 moreover comprises an equalising diaphragm 405 and a valve 406. In distinction from the embodiment according to FIG. 10, the cavity 401 of the embodiment shown in FIG. 11 includes an appropriately disposed tube 407 that is surrounded by the second cavity 401′ and which may be arranged expediently orthogonally on the axis of rotation of the gear wheel 402 in an appropriate manner, with a pressure being established in the cavity on account of bending of the tube 407 as the volume of the tube 407 changes when the tube is bent. Bending of the tube 407 may therefore be induced by an acceleration motion of the sound generator 1. The cavity 409 surrounds the tube 407 and does not communicate with the cavity 401 and may be filled with a liquid or gas. In the embodiment according to FIG. 11, the tube 407 may be expediently configured as square tube, which permits bending and response to acceleration motions as a function of the orientation. It is self-evident that the tube 407 may also be configured as round tube and that the properties of the material and the geometric dimensions and configurations may be so selected that a predetermined acceleration results in a corresponding predetermined bending of the tube 407 and therefore in the establishment of a corresponding predetermined pressure in the cavity 401. The tube 407 may be expediently provided with at least one micro weight 408 in the zone of the freely oscillating end, which weight takes an influence on the bending of the tube 407 at a predetermined acceleration, which, in its turn, takes an influence on the establishment of pressure in the cavity 401 and in the subsequent ratchet function and hence in the production of sound.

Moreover, it is possible and expedient to dispose a magnetic weight 408 on the diaphragm 404 of the embodiment 10 or on the tube 407 in the embodiment according to FIG. 11, so that acceleration or pressure may be incited or a sound may be produced in interaction with an external magnet 7 by means of electromagnetic or elector-mechanical stimulation. When such an inventive sound generator is arranged, for instance, behind a blood vessel it can be excited as has been described before. In this manner, a Doppler effect may be expediently utilised for flow metering when such an inventive sound generator is caused to vibrate by an external alternating magnetic field, i.e. when it is excited. Furthermore, the tuning of a signal becomes expediently possible. It is clear that corresponding modifications are expediently possible also in other embodiments of the present invention.

FIG. 12 illustrates a schematic view of a sound generator 1 configured in the form of a sonic thermometer 500 in accordance with the invention, which comprises a cavity 501 inside which at least one diaphragm is tensioned whilst an appropriate memory material 503 is so arranged that it hits against the diaphragm 502 when a predetermined temperature level is reached, thus generating a sound. A sound generator configured as sonic thermometer 500 in accordance with the invention may be disposed in a chain with specific advantages, with a plurality of sonic thermometers 500 being disposed one beside the other, which may be expediently present different configurations so that a great number of different temperature levels can be detected. The following Table 2 illustrates examples of various configurations of such inventive chains of sonic temperature sensors.

A succession of individual sensors permits a detection of temperature from the inside of the body with acoustic support. Clinically appropriate temperature limits may be set with sufficient precision. Hence a chain of sonic temperature sensors constitutes a sonic thermometer.

TABLE 2

| | | Tumour heating, tissue | | | | |
|---|---|---|---|---|---|---|
| | | regeneration (tendons, | General application in | Other | | |
| | Endoprosthesis | bones, . . . ) | veterinary medicine | application A | Other application B | Other application C |
| | ° C. | ° C. | ° C. | ° C. | ° C. | ° C. |
| Sensor 1 | 35 | 36.0 | 0 | 0 | 20 | 20 |
| Sensor 2 | 36 | 36.5 | 5 | 5 | 25 | 23 |
| Sensor 3 | 37 | 37.0 | 10 | 10 | 30 | 26 |
| Sensor 4 | 38 | 37.5 | 15 | 15 | 31 | 29 |
| Sensor 5 | 39 | 38.0 | 20 | 20 | 32 | 32 |
| Sensor 6 | 40 | 38.5 | 25 | 25 | 33 | 35 |
| Sensor 7 | 41 | 39.0 | 30 | 30 | 34 | 36 |
| Sensor 8 | 42 | 39.5 | 32 | 35 | 35 | 37 |
| Sensor 9 | 43 | 40.0 | 34 | 40 | 36 | 38 |
| Sensor 10 | 44 | 40.5 | 36 | 45 | 37 | 39 |
| Sensor 11 | 46 | 41.0 | 37 | 50 | 38 | 40 |
| Sensor 12 | 48 | 41.5 | 38 | 55 | 39 | 41 |
| Sensor 13 | 50 | 42.0 | 39 | 60 | 40 | 42 |
| Sensor 14 | 52 | 42.5 | 40 | 65 | 41 | 43 |
| Sensor 15 | 54 | 43.0 | 41 | 70 | 42 | 44 |
| Sensor 16 | 56 | 43.5 | 42 | 75 | 43 | 45 |
| Sensor 17 | 58 | 44.0 | 43 | 80 | 44 | 50 |
| Sensor 18 | 60 | 44.5 | 44 | 85 | 45 | 55 |
| Sensor 19 | 65 | 45.0 | 45 | 90 | 46 | 60 |
| Sensor 20 | 70 | 45.5 | 50 | 95 | 47 | 65 |

A chain of sonic generators dependent on temperature may serve to create an implantable sonic thermometer operating with a sufficiently high precision. Table 2 indicates sensible temperature intervals for specific applications in medicine or veterinary medicine.

It goes without saying that the sensors may also be arranged in a suitable bundle or cluster, for instance, rather than in a chain array. A plurality of bundles could consist, for example, of comparable chains of temperature sensors so as to provide for enhanced reliability in the generation of sound as a function of temperature. A chain of temperature sensors which generate each a specified tone in a defined range when the temperature changes may therefore permit the detection of temperature in a certain interval, for instance of 0.5 degrees.

In addition to applications in human and veterinary medicine, further applications of an inventive sonic thermometer are evident, for instance for the control of chemical reactions, even in the high temperature range. Apart therefrom, sonic thermometers could serve for the redundant detection of temperature, e.g. in aeronautical and astronautical engineering, for measuring temperatures on the outside skin of a bird or missile (safety aspect).

FIG. 13 is a schematic illustration of one embodiment of the inventive method and of the inventive system for the detection and analysis of conditions and processes inside the human or animal body, which includes a sound generator 1, an acoustic receiver unit 2, the human or animal body 3, an analyser unit 5 and the stimulating unit 6 described already with reference to FIG. 11.

In the inventive method operating on the inventive system schematically illustrated in FIG. 11, the signals emitted by the appropriately arranged sound generators are detected by suitably disposed receiver units 2 which transmit the received signals to the analyser unit 5. The analyser unit then processes the signals initially by scanning and filtering, and then a Fourier transform if realised in an appropriate manner. The analyser unit may expediently also control the stimulating unit 6 in response to the received signals so that the stimulation of the sound generators 1 will be optimised in this manner. Moreover, the frequency spectrums received by the analyser unit 5 may be compared against known frequency spectrums whereupon a lock-in process is performed. It is expediently possible in this way to perform a comparison against known physiological characteristic values whilst physiological changes are established. It is evident that the results of the analyser unit 5 may be eventually output in digital and/or graphic form, stored and displayed.

It is further expedient and possible to employ a signal amplifier in the analysis of the signals, possibly with an analog signal filtering process. The signals are appropriately processes by means of a μ-controller or DSP (digital signal processor) or PC, e.g. in combination with an USB measuring unit. In particular, the discrete Fourier transformation, DFT/FFT, if applicable also DFT/FFT with resolution in time, i.e. SFT, digital filtering and, if applicable, re-transformation are expedient techniques coming into consideration as the algorithms of analysis.

The inventive analyser unit 5 permits the evaluation of any frequency information included in the signal, also in the case of useful signals ranging extremely below of noise signals (noise amplitudes). With the Fourier transformation technique SFT (SFT: short-time four transformation, not to be confused with FFT: fast Fourier transformation) with resolution in terms of time, Fourier spectrums are represented in time-resolved form in a 3D diagram me, which means that additionally the point of time can be read by which the frequency is present.

Amplitude information of a weak character only may be detected by additional digital filtering of the Fourier spectrum with subsequent re-transformation.

Apart from the aforedescribed methods of sound generation, a hermetically encapsulated bio-compatible gas generator may generate a gas (methane, bio-compatible) and hence a gas flow on the basis of a chemical reaction (e.g. a few micro litres of water and a few micro grammes of aluminium carbide), which flow may be utilized to generate sound and for the subsequent detection of movements or blood streams. As an alternative to the gas production on the basis of a chemical reaction, it is possible that controlled bio-gas production on the basis of micro organisms such as bacteria is performed in a bio-compatible gas generator, which micro organisms produce gas in a controlled process inside an encapsulated volume.

### LIST OF REFERENCE NUMERALS

Sound generator 1
Acoustic receiver unit 2
Body 3
Body surface 31
Prosthesis, bone or blood vessel 4
Analyser unit 5
Stimulating unit 6
Whistle 100
Air stream generator 102
Lever arm 103
Diaphragm 104
Tube 105
Joint 106
Capillary system 107
Whistling-tone unit 108
Resonant cavity 109
Valve 110
Lip 111
Pulse sensor 200
Exterior shell 201
First diaphragm 202
Second diaphragm 203, 203′
Lever 204
Clapper 205
Frame 206
Chime/ratchet 300
Tube 301
Joint 302
Lever 303, 303′
Sealing lip 304
Restoring spring 305
Stop 306, 306′
Diaphragm 307
Projections 308
Prosthesis 310
Resonant volume 311
Ratchet 400
Cavity 401
Second cavity 401′
Gear wheel 402
Projections 403
Diaphragm 404
Equalising diaphragm 405
Valve 406
Tube 407
Weight/responsive material 408
Sonic thermometer 500
Cavity 501
Diaphragm 502
Memory material 503

1. Sound generator (1) for implantation into the human or animal body (3) for detection of physiologic and/or non-physiologic and/or pathologic processes in interaction with an acoustic receiver unit (2), the sound is generated from movements and/or forces and/or pressures and/or the temperature in the human or animal body (3).

2. Sound generator (1) according to claim 1, characterised in that the sound generator (1) consists of a bio-degradable material.

3. Sound generator (1) according to claim 1, characterised in that the sound generator (1) presents a miniaturised configuration.

4. Sound generator (1) according to claim 1, characterised in that the sound generator (1) is configured in the form of a whistle (100).

5. Sound generator (1) according to claim 1, characterised in that the sound generator (1) is configured in the form of a pulse sensor (200).

6. Sound generator (1) according to claim 1, characterised in that the sound generator (1) is configured in the form of a chime and/or a ratchet.

7. Sound generator (1) according to claim 1, characterised in that the sound generator (1) is configured in the form of a sonic thermometer (500).

8. Sound generator (1) according to claim 1, characterised in that the sound generator (1) comprises a resonant cavity (109).

9. Sound generator (1) according to claim 4, characterised in that said whistle (100) includes an air stream generator (102), a capillary system (107), a whistling-tone unit (108) and a resonant cavity (109), which are each so configured and disposed that a strong stream of air is generated as a result of bending of said whistle (100), which generates a defined whistling tone or clicking sound.

10. Sound generator (1) according to claim 9, characterised in that said air stream generator (102) includes a cavity with a diaphragm (104) which, when the sound generator (1) is bent, results in a volume compression of said cavity, and consists of a mechanism operating as a function of motion and changing the volume, which is constituted by a plurality of lever arms (103) which are so disposed that the volume compression of said cavity is amplified; that said cavity of said air stream generator (102) is filled with a non-compressible liquid; that a discharging tube (105) is joined to said cavity of said air stream generator (102) at the downstream end, which opens into a capillary system (107) to which a whistling-tone unit (108) is joined at the downstream side; that said capillary system (107) is configured to increase progressively in hydrophobia in a direction towards said whistling-tone unit (108) and that the region in the vicinity of said whistling-tone unit (108) is designed to be strongly hydrophobic so that it produces the effect of a liquid barrier, and that said whistling-tone unit (108) opens into a resonant cavity (109).

11. Sound generator (1) according to claim 10, characterised in that said whistling-tone comprises a tube including at least one resilient lip (11) disposed and configured in an appropriate form in said tube, which lip is caused to oscillate by the air stream generated by said air stream generator (102).

12. Sound generator (1) according to claim 10, characterised in that said capillary system (107) in the vicinity of said whistling-tone unit (108) comprises a valve (110) permitting the flow-back of air from said resonant cavity (109) into said capillary system (107).

13. Sound generator (1) according to claim 5, characterised in that said pulse sensor (200) comprises an exterior shell (201) and a first tensioned diaphragm (202) and a second diaphragm (203, 203′) on the pulse side, which is stretched over a flexible wall reinforcement, wherein a lever (204) is disposed on said diaphragm (203, 203′) on the pulse side and interacts with a clapper (205) disposed inside said exterior shell (201) in such a way that in the case of pulsation said lever operates said clapper (205) in a way that said clapper hits against said first tensioned diaphragm (202) and generates a sound.

14. Sound generator (1) according to claim 5, characterised in that said pulse sensor (200) comprises an exterior shell (201), a first tensioned diaphragm (202) and a second diaphragm (203, 203′) on the pulse side, which is stretched over a flexible wall reinforcement, wherein a lever (204) is disposed on said diaphragm (203, 203′) on the pulse side and interacts with a string disposed inside said exterior shell (201) in such a way that in the case of pulsation said lever (204) causes said string to oscillate.

15. Sound generator (1) according to claim 5, characterised in that said pulse sensor (200) with said second diaphragm (203, 203′) is disposed on a blood vessel or tissue wall inside said body (3).

16. Sound generator (1) according to claim 6, characterised in that said chime and/or ratchet (300) comprises a biased elongate tube (301) that is filled with a non-compressible liquid; and that a semihydraulic joint (302) is disposed on said tube (301) and configured in such a way that when the volume of said tube (301) is reduced, a lever (303, 303′) connected to said joint (302) generates a sound in a resonant volume (311) in which said lever (303, 303′) is disposed.

17. Sound generator (1) according to claim 16, characterised in that said lever (303, 303′) is provided with a lever end (306. 306′) which, when said joint (302) is activated, rattles over projections (308) disposed inside said resonant volume (311).

18. Sound generator (1) according to claim 16, characterised in that a diaphragm (307) is disposed on said resonant volume (311) and configured in such a way that it interacts with a stop (306, 306′) disposed on said lever (303, 303′) and generates a sound by vibration.

19. Sound generator (1) according to claim 16, characterised in that a restoring spring (305) is disposed in said resonant volume (311) and connected to said joint (302) in such a way that when the pressure in said tube (301) is reduced, the position of said ratchet (303, 303′) inside said resonant volume (311) is restored.

20. Sound generator (1) according to claim 6, characterised in that said ratchet (400) comprises a gear wheel (402) configured in a way similar to a blade wheel, which is disposed inside a cavity (401) filled with a liquid and interacts with projections (403) and a suitable means for driving said gear wheel (402), which means generates a pressure on said liquid, such that when said gear wheel is driven it rattles over said projections and generates a sound.

21. Sound generator (1) according to claim 20, characterised in that a means (403) for the equalisation of pressure of the liquid is provided in the vicinity of said gear wheel (402) and that additionally a back-flow valve (406) is provided.

22. Sound generator (1) according to claim 20, characterised in that said means for driving said gear wheel (402) and for exerting a pressure on the liquid in said cavity (401) is a cylindrical resilient diaphragm (404) disposed inside said cavity (401).

23. Sound generator (1) according to claim 22, characterised in that said cylindrical diaphragm (404) is so disposed that its cylinder axis is parallel with the axis of said gear wheel (402).

24. Sound generator (1) according to claim 20, characterised in that said means for driving said gear wheel (402) and for exerting a pressure on the liquid in said cavity (401) is an elongate extension (407) of tubular structure of said cavity (401), which is capable of freely oscillating in a second cavity (401′).

25. Sound generator (1) according to claim 24, characterised in that said tube (407) is so disposed that its axis is orthogonal on the axis of said gear wheel (402).

26. Sound generator (1) according to claim 24, characterised in that weights and/or means (408) are disposed on said tube (407) for stimulation of the vibration of said tube (407).

27. Sound generator (1) according to claim 26, characterised in that said means (408) for stimulation of the vibration of said tube (407) is a magnet interacting with an external device (6) for stimulating the vibration.

28. Sound generator (1) according to claim 7, characterised in that said sonic thermometer (500) comprises a memory material (501) that interacts with a diaphragm (502).

29. Sound generator (1) according to claim 28, characterised in that a plurality of sonic thermometers (500) is disposed in succession in the form of a chain and/or a plurality of sonic thermometers (500) is disposed one beside the other in a plane.

30. (canceled)

31. Sound generator (1) according to claim 29, characterised in that said plurality of sonic thermometers (500) is configured in different forms such that they generate a sound at predetermined different temperatures.

32. Sound generator (1) according to claim 1, characterised in that it is used to detect and analyse the pulse rate and/or movement of the cardiac wall and/or the bending and/or acceleration of body tissue.

33. Sound generator (1) according to claim 31, characterised in that it is used to detect and analyse loaded bones and/or capsular tissues and/or implants and/or prostheses and/or materials for osteosynthesis of bones.

34. System for the detection and analysis of physiologic and/or non-physiologic and/or pathologic processes, comprising at least one sound generator (1) which interacts with at least one acoustic receiver unit (2) and includes at least one analyser unit (5), the system is configured in accordance with a sound generator (1) according to claim 1.

35. Method of detecting and analysing physiologic and/or non-physiologic and/or pathologic processes by means of at least one sound generator (1) which interacts with at least one acoustic receiver unit (2) and includes at least one analyser unit (5), characterised in that in correspondence with a system according to claim 34.
